(19) United States
(12) Patent Application Publication
(10) Pub. No.: US 2010/0010256 A1
(43) Pub. Date: Jan. 14, 2010

(54) PROCESSES FOR HYDROGEN CHLORIDE OXIDATION USING OXYGEN

(75) Inventors: Andreas Bulan, Langenfeld (DE); Helmut Diekmann, Burscheid (DE); Gerhard Ruffert, Leverkusen (DE); Kaspar Hallenberger, Leverkusen (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/499,431

(22) Filed: Jul. 8, 2009

Related U.S. Application Data
(63) Continuation of application No. 11/752,598, filed on May 23, 2007, now abandoned.

(30) Foreign Application Priority Data
May 23, 2006 (DE) 10 2006 024 515.6

(51) Int. Cl. C01B 7/04; C07C 263/00; C07C 17/013

(52) U.S. Cl. 560/338; 423/507; 423/502; 570/246

(57) ABSTRACT

Processes which include: (a) providing a gas phase comprising hydrogen chloride; (b) oxidizing the hydrogen chloride in a reactor to form a product gas comprising chlorine, unreacted hydrogen chloride and water, the reactor having structural parts with inner surfaces that are contacted during oxidation by one or both of the gas phase and the product gas; (c) cooling the process gas; (d) separating the unreacted hydrogen chloride and water from the product gas; (e) drying the product gas; and (f) separating the chlorine from the product gas; wherein the inner surfaces of the reactor structural parts that are contacted during oxidation by one or both of the gas phase and the product gas are comprised of a nickel material having a nickel content of at least 60 wt. %, are described.

### BACKGROUND OF THE INVENTION

In many large-scale chemical processes, such as the production of isocyanates, in particular MDI and TDI, and in processes for the chlorination of organic substances, chlorine is used as raw material and an HCl gas stream is generally obtained as a by-product.

For the production of chlorine and, in particular, the utilization of the hydrochloric acid, for example, that is inevitably obtained in an isocyanate production process, the following different processes, which are known in principle, are mentioned here by way of example:
the production of chlorine in NaCl electrolyses and the exploitation of HCl either by selling or by further processing in oxychlorination processes, for example in the production of vinyl chloride;
the conversion of HCl to chlorine by electrolysis of aqueous HCl using diaphragms or membranes as the separation medium between the anode space and the cathode space, wherein the by-product is hydrogen;
the conversion of HCl to chlorine by electrolysis of aqueous HCl in the presence of oxygen in electrolytic cells with an oxygen depolarized cathode (ODC), wherein the by-product is water; and
the conversion of HCl gas to chlorine by gas-phase oxidation of HCl with oxygen at elevated temperatures on a catalyst, wherein the by-product is likewise water (this process has been known and used for over a century and is referred to as the “Deacon process”).

Depending on the market conditions relating to the by-products (e.g., sodium hydroxide solution, hydrogen, vinyl chloride in the first case), on the marginal conditions at the particular site in question (e.g., energy prices, integration into a chlorine infrastructure) and on the investment and operating costs, all these processes have advantages of varying importance for isocyanate production. The last-mentioned Deacon process is becoming more important.

In Deacon processes there is the problem that a chemical equilibrium between HCl, chlorine and oxygen becomes established in the reactor, which permits an HCl conversion of usually only approximately from 70 to 90%, depending on the pressure, temperature, oxygen excess, dwell time and other parameters, that is to say the process gas contains, in addition to the target product chlorine, significant proportions of unreacted HCl and significant amounts of the oxygen used in excess.

A greater technical problem in Deacon processes is the choice of the materials to be used in the various zones of the installation, because parts of the installations that come into contact with the product are attacked corrosively by the substances involved in the reaction, in particular under elevated pressure.

### BRIEF SUMMARY OF THE INVENTION

One object of the present invention is to provide a process for chlorine production by HCl oxidation which is capable of ensuring long-term operation by using specially adapted materials, and to avoid interruptions to operation owing to premature corrosion.

The invention relates, in general, to processes for carrying out an optionally catalyst-assisted hydrogen chloride oxidation process by means of oxygen. The processes generally include single- or multi-stage cooling of the process gases, separation of unreacted hydrogen chloride and water of reaction from the process gas, drying of the product gases and separation of chlorine from the mixture, wherein the parts of the process installation that come into contact with the oxidation reaction mixture are comprised of materials designed to reduce corrosion, and in particular are comprised of nickel materials.

Processes according to the present invention, by which the above-mentioned object can be achieved, include processes for carrying out an optionally catalyst-assisted hydrogen chloride oxidation process by means of oxygen, carried out in a reactor whose structural parts that come into contact with the reaction mixture are produced from nickel or an alloy containing nickel, wherein the proportion of nickel is at least 60 wt. %.

Preference is given to nickel alloys having main proportions, independently of one another, of: iron, chromium and/or molybdenum. Where only nickel is used, the proportion of nickel is particularly preferably at least 99.5 wt. %. Particular preference is given especially to materials from the group: Hastelloy® C types, Hastelloy® B types, Inconel® 600, Inconel® 625. The term “structural parts”, as used herein with reference to those parts which come into contact with the reaction mixture, refers to non-functional parts of a reactor or device, as opposed to functional parts such as catalyst materials or measuring attachments.

One embodiment of the present invention includes a process which comprises: (a) providing a gas phase comprising hydrogen chloride; (b) oxidizing the hydrogen chloride in a reactor to form a product gas comprising chlorine, unreacted hydrogen chloride and water, the reactor having structural parts with inner surfaces that are contacted during oxidation by one or both of the gas phase and the product gas; (c) cooling the process gas; (d) separating the unreacted hydrogen chloride and water from the product gas; (e) drying the product gas; and (f) separating the chlorine from the product gas; wherein the inner surfaces of the reactor structural parts that are contacted during oxidation by one or both of the gas phase and the product gas are comprised of a nickel material having a nickel content of at least 60 wt. %.

### DETAILED DESCRIPTION OF THE INVENTION

As used herein, the singular terms “a,” and “the” are synonymous and used interchangeably with “one or more” or “at least one.” Accordingly, for example, reference to “a gas” herein or in the appended claims can refer to a single gas or more than one gas. Additionally, all numerical values, unless otherwise specifically noted, are understood to be modified by the word “about.”

Reference herein to any structural part “being produced from” a material can refer to a part comprising the material (e.g., a reactor wall made of a nickel-containing alloy), being coated or lined with the material, or otherwise manufactured, designed or constructed in such a manner that the surfaces of the structural part which come into contact with a reaction phase contained therein during operation are comprised of the material.

In various embodiments of processes according to the present invention, the cooling of the product gas(es) is preferably carried out in a first heat exchanger, starting from the reactor outlet temperature to a temperature of 140 to 250° C., preferably 160 to 200° C., the structural parts of the heat exchanger that come into contact with the reaction mixture being produced from nickel or an alloy containing nickel, wherein the proportion of nickel is at least 60 wt. %. Preference is given to nickel alloys having main proportions, independently of one another, of: iron, chromium and molybdenum. Particular preference is given especially to materials selected from the group: Hastelloy® C types, Hastelloy® B types, Inconel® 600, Inconel® 625.

Preference is further given to embodiments of the present invention wherein the cooling of the product gas(es) is further carried out in a second heat exchanger, starting from the outlet temperature of the first heat exchanger to a temperature greater than or equal to 100° C., at least those structural parts of the second heat exchanger that come into contact with the reaction mixture being manufactured from a material selected from the group: steel coated with a fluoropolymer (e.g., PFA, PVDF, PTFE) and ceramics, in particular silicon carbide or silicon nitride, in particular, as tube material, particularly preferably in each case as a tube in tube bottoms, of coated steel.

Particularly preferably, the second heat exchanger is in the form of a tubular heat exchanger in which the jacket is manufactured from steel coated with fluoropolymer and the tubes of the tube bundle comprise a ceramic material, preferably silicon carbide or silicon nitride.

Very particular preference is given to those embodiments wherein a second heat exchanger is operated in such a manner that the product gas to be cooled is fed into the jacket of the heat exchanger and the cooling medium is passed through the tubes of the heat exchanger.

In a particularly preferred embodiment of a process according to the invention, the cooling of the product gas(es) is further carried out in a third heat exchanger starting from the outlet temperature of the second heat exchanger to condensation of liquid hydrochloric acid, in particular to a temperature greater than or equal to 5° C., at least those structural parts of the third heat exchanger that come into contact with the reaction mixture being manufactured from a material selected from the group: fluoropolymers, (in particular tetrafluorethylene perfluoroalkoxy vinylether copolymer (PFA), polyvinylidene fluoride (PVDF), polytetrafluorethylene (PTFE) or polyethylenecotetrafluorethylene (ETFE)), and ceramics, in particular silicon carbide or silicon nitride, in particular in each case as a tube in tube bottoms on coated steel.

In various preferred embodiments of the present invention, the product gas can be cooled in the cooling stage to less than or equal to 100° C. and then introduced for separation into an HCl absorption stage, which can be carried out using water or an aqueous solution of hydrogen chloride having a concentration of up to 30 wt. %, at least those structural parts of the HCl absorption installation that come into contact with the reaction mixture being manufactured from a material selected from the group: glass-lined steel, graphite, silicon carbide, steel coated with glass-fibre reinforced plastic (GFRP), in particular based on polyester resins or polyvinyl ester resins, or steel coated and/or lined with fluoropolymers, in particular steel optionally coated with PFA or ETFE and lined with PTFE.

The drying of the chlorine and oxygen mixture, which is preferably largely free of HCl, can be carried out in various drying apparatuses, preferably by use of concentrated sulfuric acid, in which at least those structural parts of the drying apparatuses that come into contact with the reaction mixture are manufactured from a material selected from the group: steel of the Hastelloy® C 2000 or Hastelloy® B type, Si-containing stainless steels or graphite.

The separation of the chlorine from the chlorine and oxygen mixture can particularly preferably be carried out in separating apparatuses in which at least those structural parts of the separating apparatuses that come into contact with the gas mixture are manufactured from carbon steel.

Particular preference is given also to a variant of the process that is characterised in that the liquid phase of chlorine obtained from the separation of the chlorine from the chlorine and oxygen mixture is vaporised again in a vaporising apparatus in which at least those structural parts of the vaporising apparatus that come into contact with the product are manufactured from carbon steel.

In a particularly preferred embodiments of processes according to the present invention, the hydrogen chloride of the HCl oxidation process comes from an isocyanate production process, and the purified chlorine is fed back into the isocyanate production process.

An alternative preferred process is characterised in that the hydrogen chloride of the HCl oxidation process comes from a chlorination process of organic compounds of chlorinated aromatic compounds, and the purified chlorine is fed back into the chlorination process.

In certain preferred embodiments of the present invention, the hydrogen chloride of the HCl oxidation process can be provided from both an isocyanate production process and a chlorination process of organic compounds of chlorinated aromatic compounds, and the purified chlorine obtained by the process can be recycled to either or both of the isocyanate production process and the chlorination process of organic compounds.

The various embodiments of processes according to the present invention are particularly preferably carried out in a such a manner that the HCl oxidation process takes place at a pressure of 3 to 30 bar.

Various preferred process embodiments are characterised in that the HCl oxidation process is a Deacon process, that is to say a catalysed gas-phase oxidation of HCl by means of oxygen.

In a first step of a particularly preferred process embodiment, which relates to the integration of the novel combined chlorine preparation process into an isocyanate preparation, the preparation of phosgene takes place by reaction of chlorine with carbon monoxide. The synthesis of phosgene is sufficiently well known and is described, for example, in Ullmanns Enzyklopädie der industriellen Chemie, 3rd Edition, Volume 13, pages 494-500. On an industrial scale, phosgene is predominantly produced by reaction of carbon monoxide with chlorine, preferably on activated carbon as catalyst. The strongly exothermic gas phase reaction typically takes place at a temperature of from at least 250° C. to not more than 600° C., generally in tubular reactors. The heat of reaction can be dissipated in various ways, for example by means of a liquid heat-exchange agent, as described, for example, in specification WO 03/072237 A1, the entire contents of which are incorporated herein by reference, or by vapour cooling via a secondary cooling circuit while simultaneously using the heat of reaction to produce steam, as disclosed, for example, in U.S. Pat. No. 4,764,308, the entire contents of which are incorporated herein by reference.

In a subsequent process step, at least one isocyanate is formed from the phosgene formed in the first step, by reaction with at least one organic amine or with a mixture of two or more amines. This second process step is also referred to hereinbelow as phosgenation. The reaction takes place with the formation of hydrogen chloride as by-product, which is obtained in the form of a mixture with the isocyanate.

The synthesis of isocyanates is likewise known in principle from the prior art, phosgene generally being used in a stoichiometric excess, based on the amine. The phosgenation is conventionally carried out in the liquid phase, it being possible for the phosgene and the amine to be dissolved in a solvent. Preferred solvents for the phosgenation are chlorinated aromatic hydrocarbons, such as chlorobenzene, o-dichlorobenzene, p-dichlorobenzene, trichlorobenzenes, the corresponding chlorotoluenes or chloroxylenes, chloroethylbenzene, monochlorodiphenyl, α- or β-naphthyl chloride, benzoic acid ethyl ester, phthalic acid dialkyl esters, diisodiethyl phthalate, toluene and xylenes. Further examples of suitable solvents are known in principle from the prior art. As is additionally known from the prior art, for example according to specification WO 96/16028, the entire contents of which are incorporated herein by reference, the resulting isocyanate itself can also serve as the solvent for phosgene. In another, preferred embodiment, the phosgenation, in particular of suitable aromatic and aliphatic diamines, takes place in the gas phase, that is to say above the boiling point of the amine. Gas-phase phosgenation is described, for example, in EP 570 799 A1, the entire contents of which are incorporated herein by reference. Advantages of this process over liquid-phase phosgenation, which is otherwise conventional, are the energy saving, which results from the minimisation of a complex solvent and phosgene circuit.

Suitable organic amines are in principle any primary amines having one or more primary amino groups which are able to react with phosgene to form one or more isocyanates having one or more isocyanate groups. The amines have at least one, preferably two, or optionally three or more primary amino groups. Accordingly, suitable organic primary amines are aliphatic, cycloaliphatic, aliphatic-aromatic, aromatic amines, diamines and/or polyamines, such as aniline, halo-substituted phenylamines, for example 4-chlorophenylamine, 1,6-diaminohexane, 1-amino-3,3,5-trimethyl-5-amino-cyclohexane, 2,4-, 2,6-diaminotoluene or mixtures thereof, 4,4′-, 2,4′- or 2,2′-diphenylmethanediamine or mixtures thereof, as well as higher molecular weight isomeric, oligomeric or polymeric derivatives of the mentioned amines and polyamines. Further possible amines are known in principle from the prior art. Preferred amines for the present invention are the amines of the diphenylmethanediamine group (monomeric, oligomeric and polymeric amines), 2,4-, 2,6-diaminotoluene, isophoronediamine and hexamethylenediamine. In the phosgenation, the corresponding isocyanates diisocyanatodiphenylmethane (MDI, monomeric, oligomeric and polymeric derivatives), toluylene diisocyanate (TDI), hexamethylene diisocyanate (HDI) and isophorone diisocyanate (IPDI) are obtained.

The amines can be reacted with phosgene in a single-stage or two-stage or, optionally, a multi-stage reaction. Both a continuous and a discontinuous procedure are possible.

If a single-stage phosgenation in the gas phase is chosen, the reaction is carried out above the boiling temperature of the amine, preferably within a mean contact time of from 0.5 to 5 seconds and at a temperature of from 200 to 600° C.

Phosgenation in the liquid phase is conventionally carried out at a temperature of from 20 to 240° C. and a pressure of from 1 to about 50 bar. Phosgenation in the liquid phase can be carried out in a single stage or in a plurality of stages, it being possible to use phosgene in a stoichiometric excess. The amine solution and the phosgene solution are combined via a static mixing element and then guided through one or more reaction columns, for example from bottom to top, where the mixture reacts completely to form the desired isocyanate. In addition to reaction columns provided with suitable mixing elements, reaction vessels having a stirrer device can also be used. As well as static mixing elements, it is also possible to use special dynamic mixing elements. Suitable static and dynamic mixing elements are known in principle from the prior art.

In general, continuous liquid-phase isocyanate production on an industrial scale is carried out in two stages. In the first stage, generally at a temperature of not more than 220° C., preferably not more than 160° C., the carbamoyl chloride is formed from amine and phosgene and amine hydrochloride is formed from amine and cleaved hydrogen chloride. This first stage is highly exothermic. In the second stage, both the carbamoyl chloride is cleaved to isocyanate and hydrogen chloride and the amine hydrochloride is reacted to carbamoyl chloride. The second stage is generally carried out at a temperature of at least 90° C., preferably from 100 to 240° C.

After the phosgenation, the isocyanates formed in the phosgenation are separated off in a third step. This is effected by first separating the reaction mixture of the phosgenation into a liquid and a gaseous product stream in a manner known in principle to the person skilled in the art. The liquid product stream contains substantially the isocyanate or isocyanate mixture, the solvent and a small part of unreacted phosgene. The gaseous product stream consists substantially of hydrogen chloride gas, phosgene in stoichiometric excess, and small amounts of solvent and inert gases, such as, for example, nitrogen and carbon monoxide. Furthermore, the liquid stream is then conveyed to a working-up step, preferably working up by distillation, wherein phosgene and the solvent for the phosgenation are separated off in succession. In addition, further working up of the resulting isocyanates is optionally carried out, for example by fractionating the resulting isocyanate product in a manner known to the person skilled in the art.

The hydrogen chloride obtained in the reaction of phosgene with an organic amine generally contains organic minor constituents, which can be disruptive in both thermal catalysed and non-thermal activated HCl oxidation, These organic constituents include, for example, the solvents used in the isocyanate preparation, such as chlorobenzene, o-dichlorobenzene or p-dichlorobenzene.

Separation of the hydrogen chloride is preferably carried out by first separating phosgene from the gaseous product stream. Phosgene can be separated off by liquefying phosgene, for example in one or more condensers arranged in series. The liquefaction is preferably carried out at a temperature in the range of from −15 to −40° C., depending on the solvent used. By means of this deep-freezing it is additionally possible to remove portions of the solvent residues from the gaseous product stream.

Additionally or alternatively, the phosgene can be washed out of the gas stream in one or more stages using a cold solvent or solvent/phosgene mixture. Suitable solvents therefor are, for example, the solvents chlorobenzene and o-dichlorobenzene already used in the phosgenation. The temperature of the solvent or of the solvent/phosgene mixture is in the range from −15 to −46° C.

The phosgene separated from the gaseous product stream can be fed back to the phosgenation. The hydrogen chloride obtained after separating off the phosgene and part of the solvent residue can contain, in addition to inert gases such as nitrogen and carbon monoxide, also from 0.1 to 1 wt. % solvent and from 0.1 to 2 wt. % phosgene.

Purification of the hydrogen chloride is then optionally carried out in order to reduce the content of traces of solvent. This can be effected, for example, by means of separation by freezing, where the hydrogen chloride is passed, for example, through one or more cold traps, depending on the physical properties of the solvent.

In particularly preferred embodiments of the hydrogen chloride purification that is optionally provided, the stream of hydrogen chloride flows through two heat exchangers connected in series, the solvent to be removed being separated out by freezing at, for example, −40° C., depending on the fixed point. The heat exchangers are preferably operated alternately, the solvent previously separated out by freezing being thawed by the gas stream in the heat exchanger that is passed through first. The solvent can be used again for the preparation of a phosgene solution. In the second, downstream heat exchanger, which is supplied with a conventional heat-exchange medium for refrigerating machines, for example a compound from the group of the Freons, the gas is cooled to preferably below the fixed point of the solvent, so that the latter crystallises out, When the thawing and crystallisation operation is complete, the gas stream and the cooling agent stream are changed over, so that the function of the heat exchangers is reversed. In this manner, the solvent content of the hydrogen-chloride-containing gas stream can be reduced to preferably not more than 500 ppm, particularly preferably not more than 50 ppm, very particularly preferably to not more than 20 ppm.

Alternatively, the purification of the hydrogen chloride can be carried out preferably in two heat exchangers connected in series, for example according to U.S. Pat. No. 6,719,957. The hydrogen chloride is thereby preferably compressed to a pressure of from 5 to 20 bar, preferably from 10 to 15 bar, and the compressed gaseous hydrogen chloride is fed at a temperature of from 20 to 60° C., preferably from 30 to 50° C., to a first heat exchanger, where the hydrogen chloride is cooled with cold hydrogen chloride having a temperature of from −10 to −30° C. from a second heat exchanger. Organic constituents condense thereby and can be fed to disposal or re-use. The hydrogen chloride passed into the first heat exchanger leaves it at a temperature of from −20 to 0° C. and is cooled in the second heat exchanger to a temperature of from −10 to −30° C. The condensate formed in the second heat exchanger consists of further organic constituents as well as small amounts of hydrogen chloride. In order to avoid losing hydrogen chloride, the condensate leaving the second heat exchanger is fed to a separating and vaporising unit. This can be a distillation column, for example, in which the hydrogen chloride is driven out of the condensate and fed back into the second heat exchanger. It is also possible to feed the hydrogen chloride that has been driven out back into the first heat exchanger. The hydrogen chloride cooled and freed of organic constituents in the second heat exchanger is passed into the first heat exchanger at a temperature of from −10 to −30° C. After heating to from 10 to 30° C., the hydrogen chloride freed of organic constituents leaves the first heat exchanger.

In certain process embodiments, which are likewise preferred, the optional purification of the hydrogen chloride of organic impurities, such as solvent residues, can take place on activated carbon by means of adsorption. In such processes, for example, the hydrogen chloride, after removal of excess phosgene, is passed over or through bulk activated carbon at a pressure difference of from 0 to 5 bar, preferably from 0.2 to 2 bar. The flow velocity and the dwell time are thereby adapted to the content of impurities in a manner known to the person skilled in the art. The adsorption of organic impurities on other suitable adsorbents, for example on zeolites, is also possible.

In a further process embodiment, which is also preferred, distillation of the hydrogen chloride can be provided for the optional purification of the hydrogen chloride from the phosgenation. This is carried out after condensation of the gaseous hydrogen chloride from the phosgenation. In the distillation of the condensed hydrogen chloride, the purified hydrogen chloride is removed as the first fraction of the distillation, the distillation being carried out under conditions of pressure, temperature, etc. that are known to the person skilled in the art and are conventional for such a distillation.

The hydrogen chloride separated and optionally purified according to the processes described above can subsequently be fed to the HCl oxidation using oxygen.

As already described above, a catalytic process referred to as the Deacon process is preferably used for HCl oxidation according to the present invention. In such processes, hydrogen chloride is oxidized with oxygen in an exothermic equilibrium reaction to give chlorine, with the formation of water vapor. The reaction temperature can be 150 to 500° C., and the reaction pressure can be 1 to 25 bar. Because this is an equilibrium reaction, it is advantageous to work at the lowest possible temperatures at which the catalyst still exhibits sufficient activity. Furthermore, it is advantageous to use oxygen in more than stoichiometric amounts, based on the hydrogen chloride. A two- to four-fold oxygen excess, for example, can be used. Because there is no risk of selectivity losses, it can be economically advantageous to work at a relatively high pressure and accordingly with a longer dwell time compared with normal pressure.

Suitable preferred catalysts for the Deacon process contain ruthenium oxide, ruthenium chloride or other ruthenium compounds on silicon dioxide, aluminium oxide, titanium dioxide or zirconium dioxide as support. Suitable catalysts can be obtained, for example, by applying ruthenium chloride to the support and then drying or drying and calcining. In addition to or instead of a ruthenium compound, suitable catalysts can also contain compounds of different noble metals, for example gold, palladium, platinum, osmium, iridium, silver, copper or rhenium. Suitable catalysts can also contain chromium(III) oxide.

The catalytic oxidation of hydrogen chloride can be carried out adiabatically or, preferably, isothermally or approximately isothermally, discontinuously, but preferably continuously, as a fluidised or fixed bed process, preferably as a fixed bed process, particularly preferably in tubular reactors on heterogeneous catalysts at a reactor temperature of from 180 to 500° C., preferably from 200 to 400° C., particularly preferably from 220 to 350° C., and a pressure of from 1 to 25 bar (from 1000 to 25,000 hPa), preferably from 1.2 to 20 bar, particularly preferably from 1.5 to 17 bar and especially from 2.0 to 15 bar.

Reaction apparatuses in which the catalytic oxidation of hydrogen chloride can be carried out include fixed bed or fluidised bed reactors. The catalytic oxidation of hydrogen chloride can preferably also be carried out in a plurality of stages.

In the case of the isothermal or approximately isothermal procedure, it is also possible to use a plurality of reactors, that is to say from 2 to 10, preferably from 2 to 6, particularly preferably from 2 to 5, especially from 2 to 3 reactors, connected in series with additional intermediate cooling. The oxygen can be added either in its entirety, together with the hydrogen chloride, upstream of the first reactor, or distributed over the various reactors. This series connection of individual reactors can also be combined in one apparatus.

A further preferred embodiment of a device suitable for the process includes using a structured bulk catalyst in which the catalytic activity increases in the direction of flow. Such structuring of the bulk catalyst can be effected by variable impregnation of the catalyst support with active substance or by variable dilution of the catalyst with an inert material. There can be used as the inert material, for example, rings, cylinders or spheres of titanium dioxide, zirconium dioxide or mixtures thereof, aluminium oxide, steatite, ceramics, glass, graphite or stainless steel. In the case of the use of catalyst shaped bodies, which is preferred, the inert material should preferably have similar outside dimensions.

Suitable catalyst shaped bodies include shaped bodies of any shape, preferred shapes being lozenges, rings, cylinders, stars, cart wheels or spheres and particularly preferred shapes being rings, cylinders or star-shaped extrudates.

Suitable heterogeneous catalysts include in particular ruthenium compounds or copper compounds on support materials, which can also be doped, with preference being given to optionally doped ruthenium catalysts. Examples of suitable support materials are silicon dioxide, graphite, titanium dioxide of rutile or anatase structure, zirconium dioxide, aluminium oxide or mixtures thereof, preferably titanium dioxide, zirconium dioxide, aluminium oxide or mixtures thereof, particularly preferably γ- or δ-aluminium oxide or mixtures thereof.

The copper or ruthenium supported catalysts can be obtained, for example, by impregnating the support material with aqueous solutions of CuCl₂ or RuCl₃ and optionally of a promoter for doping, preferably in the form of their chlorides. Shaping of the catalyst can take place after or, preferably, before the impregnation of the support material.

Suitable promoters for the doping of the catalysts include alkali metals such as lithium, sodium, potassium, rubidium and caesium, preferably lithium, sodium and potassium, particularly preferably potassium, alkaline earth metals such as magnesium, calcium, strontium and barium, preferably magnesium and calcium, particularly preferably magnesium, rare earth metals such as scandium, yttrium, lanthanum, cerium, praseodymium and neodymium, preferably scandium, yttrium, lanthanum and cerium, particularly preferably lanthanum and cerium, or mixtures thereof.

The shaped bodies can then be dried and optionally calcined at a temperature of from 100 to 400° C., preferably from 100 to 300° C., for example, under a nitrogen, argon or air atmosphere. The shaped bodies are preferably first dried at from 100 to 150° C. and then calcined at from 200 to 400° C.

The hydrogen chloride conversion in a single pass can preferably be limited to from 15 to 90%, preferably from 40 to 85%, particularly preferably from 50 to 70%. After separation, all or some of the unreacted hydrogen chloride can be fed back into the catalytic hydrogen chloride oxidation. The volume ratio of hydrogen chloride to oxygen at the entrance to the reactor is preferably from 1:1 to 20:1, more preferably from 2:1 to 8:1, particularly preferably from 2:1 to 5:1.

The heat of reaction of the catalytic hydrogen chloride oxidation can advantageously be used to produce high-pressure steam. This can be used, for example, to operate a phosgenation reactor and/or distillation columns, in particular isocyanate distillation columns.

The following examples are for reference and do not limit the invention described herein.

### EXAMPLES

### Example 1

Only the main components of the process streams are mentioned in the example.

For the oxidation of hydrogen chloride, a mixture of

| | |
|---|---|
| nitrogen | 1.3 t/h |
| oxygen | 15.7 t/h |
| hydrogen chloride | 35.9 t/h |
| carbon dioxide | 1.6 t/h |

is fed at a temperature of 320° C. and a pressure of 4.3 bar to a reactor in which the hydrogen chloride is reacted, on a catalyst, with oxygen to give chlorine and water. In the reactor, all the structural parts are made of the material carbon steel, which is provided with coatings and plating of nickel (purity 99.5 wt. % Ni). A process gas having the following composition:

| | |
|---|---|
| nitrogen | 1.3 t/h |
| oxygen | 9.0 t/h |
| hydrogen chloride | 5.4 t/h |
| carbon dioxide | 1.7 t/h |
| chlorine | 30.4 t/h |
| water | 8.3 t/h |

leaves the reactor at a temperature of 333° C. and 3.4 bar. This process gas stream is passed to a first heat exchanger, the structural parts of which that come into contact with product are manufactured from nickel purity 99.5 wt. % Ni). The material is present partly in the form of a lining and partly in solid form. The process gas is thereby cooled to 250° C.

In a second heat exchanger, the structural parts of which that come into contact with product are manufactured from silicon carbide. Also provided are ceramics tubes (of silicon carbide), which are connected to PTFE-coated tube plates and are constructed in the form of a heat exchanger. The process gas stream is cooled therein to 100° C.; the pressure is 3.15 bar.

This process gas is passed to a HCl absorption installation for removal of hydrogen chloride and water. The HCl absorption installation has the following construction:
HCl and H₂O in the crude gas are removed in an absorption column. To that end, the crude gas is introduced above the bottom. Water is applied at the top of the column. HCl and H₂O are obtained in the bottom in the form of 25 wt. % hydrochloric acid; the purified crude gas at the top of the column contains O₂ and Cl₂ and is saturated with water vapour.

In order to increase the oxygen conversion and to dissipate the heat of absorption that forms, 25 wt. % hydrochloric acid is pumped from the bottom to the top of the column. The circulated hydrochloric acid is cooled by means of a heat exchanger.

The parts of the hydrogen chloride absorption installation that come into contact with product consist of components lined with plastics material (PVDF).

A gas stream having the following composition:

| | |
|---|---|
| nitrogen | 1.3 t/h |
| oxygen | 9.0 t/h |
| carbon dioxide | 1.7 t/h |
| chlorine | 30.4 t/h |

can be removed from the hydrogen chloride absorption. The temperature is 25° C. and the pressure is 3.0 bar. In order to remove traces of water, the process gas is dried with sulfuric acid. The drying is carried out by means of a drying column. The Cl₂/O₂ gas mixture saturated with water vapour is passed into the column above the bottom. 98 wt. % sulfuric acid is applied at the top of the column. The mass transport of the water vapour into the sulfuric acid takes place in the column. The sulfuric acid diluted to approximately 75 to 78 wt. % is discharged at the bottom of the column.

The structural parts of the drying device that come into contact with product are made of carbon steel.

The dried process gas stream is compressed to 11.9 bar, and the chlorine gas present therein is liquefied.

After compression, the gas is cooled recuperatively to −45° C. Inert substances (O₂, CO₂) are stripped off in a distillation column. Liquid chlorine is obtained at the bottom. Chlorine is then vaporised and thereby cools the compressed Cl₂/O₂ gas mixture.

All parts of the chlorine liquefaction device that come into contact with product are manufactured from carbon steel. The chlorine removed from the chlorine liquefaction, 29.4 t/h, 11.6 bar, 35° C., which still contains small amounts of carbon dioxide (0.15 t/h), is fed to a storage tank. Part of the residual gas that remains, consisting of

| | |
|---|---|
| nitrogen | 1.3 t/h |
| oxygen | 9.0 t/h |
| carbon dioxide | 1.55 t/h |
| chlorine | 1.0 t/h, |

is discarded and the remainder, consisting of

| | |
|---|---|
| nitrogen | 0.96 t/h |
| oxygen | 6.4 t/h |
| carbon dioxide | 1.1 t/h |
| chlorine | 0.73 t/h, |

is added to the gases fed to the reactor. Corrosion and wear of the apparatus are reduced by the combination of apparatus materials chosen in the different process zones.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A process comprising:
(a) providing a gas phase comprising hydrogen chloride;
(b) oxidizing the hydrogen chloride in a reactor to form a product gas comprising chlorine, unreacted hydrogen chloride, oxygen and water, the reactor having structural parts with inner surfaces that are contacted during oxidation by one or both of the gas phase and the product gas;
(c) cooling the process gas;
(d) separating the unreacted hydrogen chloride and water from the product gas;
(e) drying the product gas; and
(f) separating the chlorine from the product gas;
wherein the inner surfaces of the reactor structural parts that are contacted during oxidation by one or both of the gas phase and the product gas are comprised of a nickel material having a nickel content of at least 60 wt. %.

2. The process according to claim 1, wherein cooling the product gas comprises introducing the product gas into a first heat exchanger having structural parts with inner surfaces that are contacted by the product gas during cooling, wherein the product gas exits the first heat exchanger at a temperature of 140 to 250° C., and wherein the inner surfaces of the first heat exchanger structural parts that are contacted by the product gas during cooling are comprised of a nickel material having a nickel content of at least 60 wt. %.

3. The process according to claim 2, wherein cooling the product gas further comprises introducing the product gas, after exiting the first heat exchanger, into a second heat exchanger having structural parts with inner surfaces that are contacted by the product gas during cooling, wherein the product gas exits the second heat exchanger at a temperature greater than or equal to 100° C., and wherein the inner surfaces of the second heat exchanger structural parts that are contacted by the product gas during cooling are comprised of a material selected from the group consisting of fluoropolymers, ceramics and combinations thereof.

4. The process according to claim 3, wherein cooling the product gas further comprises introducing the product gas, after exiting the second heat exchanger, into a third heat exchanger having structural parts with inner surfaces that are contacted by the product gas during cooling, wherein cooling is carried out to condensation of liquid hydrochloric acid, and wherein the inner surfaces of the third heat exchanger structural parts that are contacted by the product gas during cooling are comprised of a material selected from the group consisting of fluoropolymers, ceramics and combinations thereof.

5. The process according to claim 1, wherein cooling the product gas is carried out to a product gas temperature less than or equal to 100° C., and wherein separating the unreacted hydrogen chloride and water from the product gas is carried out in an HCl absorption installation using water or an aqueous solution of hydrogen chloride having a HCl concentration of up to 30 wt. %; the HCl absorption installation having structural parts with inner surfaces that are contacted during separation by one or more of the product gas, the unreacted hydrogen chloride and water, wherein the inner surfaces of the HCl absorption installation that are contacted during separation by one or more of the product gas, the unreacted hydrogen chloride and water are comprised of a material selected from the group consisting of glass-lined steel, graphite, silicon carbide, glass-fiber reinforced plastic-coated steel, fluoropolymer-coated steel, and combinations thereof.

6. The process according to claim 3, wherein cooling the product gas is carried out to a product gas temperature less than or equal to 100° C., wherein separating the
unreacted hydrogen chloride and water from the product gas is carried out in an HCl absorption installation using water or an aqueous solution of hydrogen chloride having a HCl concentration of up to 30 wt. %; the HCl absorption installation having structural parts with inner surfaces that are contacted during separation by one or more of the product gas, the unreacted hydrogen chloride and water, wherein the inner surfaces of the HCl absorption installation that are contacted during separation by one or more of the product gas, the unreacted hydrogen chloride and water are comprised of a material selected from the group consisting of glass-lined steel, graphite, silicon carbide, glass-fiber reinforced plastic-coated steel, fluoropolymer-coated steel, fluoropolymer-lined steel, and combinations thereof.

7. The process according to claim 1, wherein drying the product gas is carried out in a drying apparatus having structural parts with inner surfaces that are contacted by the product gas during drying, wherein the inner surfaces of the drying apparatus that are contacted by the product gas during drying are comprised of a material selected from the group consisting of Hastelloy® C 2000 steel alloys, Hastelloy® B steel alloys, Si-containing stainless steels, graphite and combinations thereof.

8. The process according to claim 4, wherein drying the product gas is carried out in a drying apparatus having structural parts with inner surfaces that are contacted by the product gas during drying, wherein the inner surfaces of the drying apparatus that are contacted by the product gas during drying are comprised of a material selected from the group consisting of Hastelloy® C 2000 steel alloys, Hastelloy® B steel alloys, Si-containing stainless steels, graphite and combinations thereof.

9. The process according to claim 5, wherein drying the product gas is carried out in a drying apparatus having structural parts with inner surfaces that are contacted by the product gas during drying, wherein the inner surfaces of the drying apparatus that are contacted by the product gas during drying are comprised of a material selected from the group consisting of Hastelloy® C 2000 steel alloys, Hastelloy® B steel alloys, Si-containing stainless steels, graphite and combinations thereof.

10. The process according to claim 1, wherein separating the chlorine from the product gas is carried out in a separating apparatus having structural parts with inner surfaces that are contacted by one or both of the product gas and the chlorine during separation, wherein the inner surfaces of the separating apparatus that are contacted by one or both of the product gas and the chlorine during separation are comprised of carbon steel.

11. The process according to claim 1, wherein the chlorine separated from the product gas comprises liquid chlorine, and wherein the process further comprises vaporizing the liquid chlorine in a vaporizing apparatus having structural parts with inner surfaces that are contacted by the chlorine during vaporization, wherein the inner surfaces of the vaporizing apparatus that are contacted by the chlorine during vaporization are comprised of carbon steel.

12. The process according to claim 10, wherein the chlorine separated from the product gas comprises liquid chlorine, and wherein the process further comprises vaporizing the liquid chlorine in a vaporizing apparatus having structural parts with inner surfaces that are contacted by the chlorine during vaporization, wherein the inner surfaces of the vaporizing apparatus that are contacted by the chlorine during vaporization are comprised of carbon steel.

13. The process according to claim 3, wherein the second heat exchanger comprises a tubular heat exchanger having: (i) a jacket comprised of fluoropolymer-coated steel and (ii) a tube bundle comprising one or more tubes comprised of a ceramic.

14. The process according to claim 6, wherein the second heat exchanger comprises a tubular heat exchanger having: (i) a jacket comprised of fluoropolymer-coated steel and (ii) a tube bundle comprising one or more tubes comprised of a ceramic.

15. The process according to claim 8, wherein the second heat exchanger comprises a tubular heat exchanger having: (i) a jacket comprised of fluoropolymer-coated steel and (ii) a tube bundle comprising one or more tubes comprised of a ceramic.

16. The process according to claim 13, wherein the process gas is introduced into the jacket of the tubular heat exchanger and a cooling medium is passed through the tube bundle of the tubular heat exchanger.

17. The process according to claim 1, wherein the oxidation of hydrogen chloride is carried out in the presence of a gas-phase oxidation catalyst.

18. The process according to claim 1, wherein at least a portion of the hydrogen chloride to be oxidized is supplied from an isocyanate production process, and at least a portion of the chlorine separated from the product gas is fed back into the isocyanate production process.

19. The process according to claim 1, wherein at least a portion of the hydrogen chloride to be oxidized is supplied from a chlorination process of organic compounds, and at least a portion of the chlorine separated from the product gas is fed back into the chlorination process.

20. The process according to claim 1, wherein the oxidation is carried out at a pressure of 3 to 30 bar.
